(19) United States
(12) Patent Application Publication
(10) Pub. No.: US 2026/0115533 A1
(43) Pub. Date: Apr. 30, 2026

(54) METHOD AND SYSTEM FOR IMPLEMENTING BOXING MOTION ANALYSIS

(71) Applicant: National Tsing Hua University, Hsinchu City (TW)

(72) Inventors: Yun-Ju LEE, Hsinchu City (TW); Chun-Yao WANG, Hsinchu City (TW); Kai-Po HSU, Hsinchu City (TW); Yu-Cheng LEE, Hsinchu City (TW)

(73) Assignee: National Tsing Hua University, Hsinchu City (TW)

(21) Appl. No.: 19/180,755

(22) Filed: Apr. 16, 2025

(30) Foreign Application Priority Data
Oct. 25, 2024 (TW) 113140770

(51) Int. Cl. A63B 24/00

(52) U.S. Cl. CPC A63B 24/0006; A63B 2220/44; A63B 2220/53; A63B 2220/836; A63B 2244/102

(57) ABSTRACT

A method for implementing boxing motion analysis includes: obtaining, from an inertial measurement unit (IMU) on a user, acceleration data at a plurality of time instances within a time period; performing a dynamic adjustment operation on the acceleration data to obtain adjusted acceleration data; performing a signal cutting operation based on the adjusted acceleration data, three average acceleration values when the IMU is in a stationary state, and three threshold values, so as to obtain a plurality of time segments each associated with one swing motion; and performing, with respect to each of the time segments, a quaternion calculation, so as to obtain rotational acceleration data, performing a first integration operation and a second integration operation to obtain a plurality of locations of the IMU over the time segment, and obtaining a trajectory of the IMU in the time segment.

### CROSS-REFERENCE TO RELATED APPLICATION

This application claims priority to Taiwanese Invention patent application No. 113140770, filed on Oct. 25, 2024, the entire disclosure of which is incorporated by reference herein.

### FIELD

The disclosure relates to a method and a system for implementing analysis, more particularly to a method and system for implementing boxing motion analysis.

### BACKGROUND

In the field of motion detection, the movements of users may be detected by wearable devices (which may include inertial measurement units (IMU)) worn by the users and recorded in the form of detection signals, which may then be processed using, for example, a computer processor.

One application of motion detection involves implementing boxing motion analysis, which may be done in situations such as training, playing games related to motion detection, mixed reality (MR), etc. Specifically, three separate wearable devices may be placed on different body parts (e.g., an arm, a forearm and a wrist) of a user. As the user performs the boxing motions, the detection signals obtained by the wearable devices may be processed, and different parameters related to the boxing motion of the user such as a punching speed, a punch count, etc., may be calculated.

### SUMMARY

It may be desirable to obtain the detection signals using less hardware components. Additionally, it may be desirable to obtain more results using the detection signals.

Therefore, an object of the disclosure is to provide a method that can alleviate at least one of the drawbacks of the prior art.

According to one embodiment of the disclosure, the method for implementing boxing motion analysis is implemented using an inertial measurement unit (IMU) placed on a wrist of a user, and a computer device connected to the IMU. The method includes:
a) obtaining, by the computer device from the IMU, plural entries of acceleration data at a plurality of time instances within a time period, each of the plural entries of acceleration data being with respect to one of three directions of a three-dimensional (3D) coordinate system;
b) performing a dynamic adjustment operation on the plural entries of acceleration data, so as to obtain a number of entries of adjusted acceleration data, wherein the dynamic adjustment operation includes implementing matrix operations on the plural entries of acceleration data using the following equation

$${A}_{t}^{O} = [ \begin{matrix}{k}_{x} & 0 & 0 \\ 0 & {k}_{y} & 0 \\ 0 & 0 & {k}_{z}\end{matrix} ] [ \begin{matrix}1 & - {θ}_{yz} & {θ}_{zy} \\ 0 & 1 & - {θ}_{zx} \\ 0 & 0 & 1\end{matrix} ] ( {A}_{t}^{s} - [ \begin{matrix}{b}_{x} & 0 & 0 \\ 0 & {b}_{y} & 0 \\ 0 & 0 & {b}_{z}\end{matrix} ] )$$

where

$${A}_{t}^{O}$$

represents a set or three entries of adjusted acceleration data with respect to the three directions at the time instance t, kₓ, k_{y} and k_{z} represent three scale factors with respect to the three directions, respectively, θ_{yz}, θ_{zy} and θ_{zx} represent three inclination factors each with respect to two of the three directions, bₓ, by and by represent three deviation factors with respect to the three directions, respectively, and

$${A}_{t}^{s}$$

represents a set of three entries of acceleration data with respect to the three directions at the time instance t;
c) performing a signal cutting operation on the plural entries of acceleration data based on the number of entries of adjusted acceleration data, three average acceleration values corresponding respectively to the three directions when the IMU is in a stationary state, and three threshold values, so as to obtain a plurality of time segments, each of the time segments being associated with one swing motion and including three subsets of accelerations with respect to the three directions, respectively; and
d) performing, with respect to each of the time segments, a quaternion calculation on the subsets of accelerations, so as to obtain plural entries of rotational acceleration data that correspond with the time instances within the time segment, respectively, performing a first integration operation on the plural entries of rotational acceleration data to obtain a plurality of velocities of the IMU over the time segment, performing a second integration operation on the plural entries of rotational acceleration data to obtain a plurality of locations of the IMU over the time segment, and obtaining a trajectory of the IMU in the time segment within the 3D coordinate system.

Another object of the disclosure is to provide a system that is configured to implement the above-mentioned method.

According to one embodiment of the disclosure, an inertial measurement unit (IMU) placed on a wrist of a user and a computer device connected to the IMU, wherein the computer device is programmed to:
obtain from the IMU plural entries of acceleration data at a plurality of time instances within a time period, each of the plural entries of acceleration data being with respect to one of three directions of a three-dimensional (3D) coordinate system;
perform a dynamic adjustment operation on the plural entries of acceleration data, so as to obtain a number of entries of adjusted acceleration data,
perform a signal cutting operation on the plural entries of acceleration data based on the number of entries of adjusted acceleration data, three average acceleration values corresponding respectively to the three directions when the IMU is in a stationary state, and three threshold values, so as to obtain a plurality of time segments, each of the time segments being associated with one swing motion and including three subsets of accelerations with respect to the three directions, respectively; and
perform, with respect to each of the time segments, a quaternion calculation on the subsets of accelerations, so as to obtain plural entries of rotational acceleration data that correspond with the time instances within the time segment, respectively, performing a first integration operation on the plural entries of rotational acceleration data to obtain a plurality of velocities of the IMU over the time segment, performing a second integration operation on the plural entries of rotational acceleration data to obtain a plurality of locations of the IMU over the time segment, and obtaining a trajectory of the IMU in the time segment within the 3D coordinate system.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a system for implementing boxing motion analysis according to one embodiment of the disclosure.

FIG. 2 is a flow chart illustrating steps of an exemplary method for implementing boxing motion analysis according to one embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “coupled to” or “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

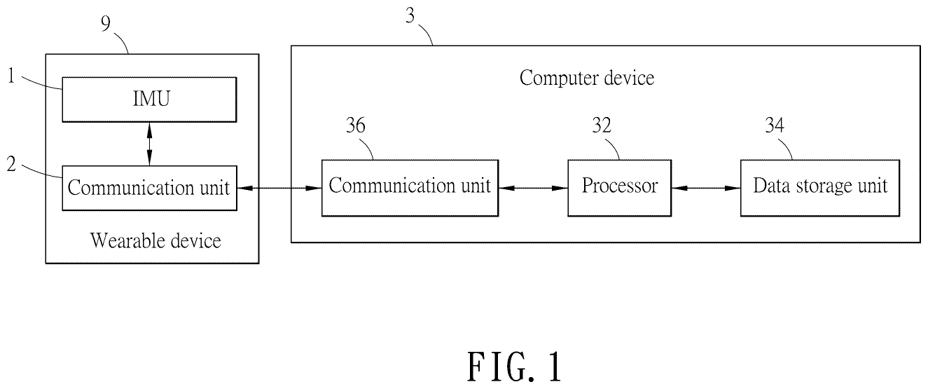

FIG. 1 is a block diagram illustrating a system for implementing boxing motion analysis according to one embodiment of the disclosure. In this embodiment, the system includes a communication unit 2, an inertial measurement unit (IMU) 1, and a computer device 3.

The IMU 1 may be embodied using a commercially available IMU, and is to be placed on a body part of a user. In this embodiment, the IMU 1 may be incorporated into a wearable device 9 (e.g., a wristband, a boxing glove, etc.), and placed in a specific location of the wearable device 9 such that, when the wearable device 9 is worn by the user on a hand, the IMU 1 is located near the wrist of the user.

The communication unit 2 is connected to the IMU 1, and may be in communication with the computer device 3. In use, the communication unit 2 may include one or more of a radio-frequency integrated circuit (RFIC), a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G) of, the fourth generation (4G) of or the fifth generation (5G) of wireless mobile telecommunications technology, or the like.

The computer device 3 may be embodied using a personal computer, a laptop, a tablet, a smartphone, a smart wearable device or other suitable electronic devices. The computer device 3 includes a processor 32, a data storage unit 34, and a communication unit 36.

The processor 32 that may be embodied using a central processing unit (CPU), a microprocessor, a microcontroller, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc.

The data storage unit 34 may be embodied using, for example, random access memory (RAM), read only memory (ROM), programmable ROM (PROM), firmware, flash memory, etc. In this embodiment, the data storage unit 34 stores a software application therein. The software application includes instructions that, when executed by the processor 32, causes the processor 32 to perform the operations as described in the subsequent paragraphs.

The communication unit 36 may be embodied using components that are similar to the communication unit 2.

In use, after the user wears the wearable device 9 and starts performing the boxing motions, a method for implementing boxing motion analysis may be initiated.

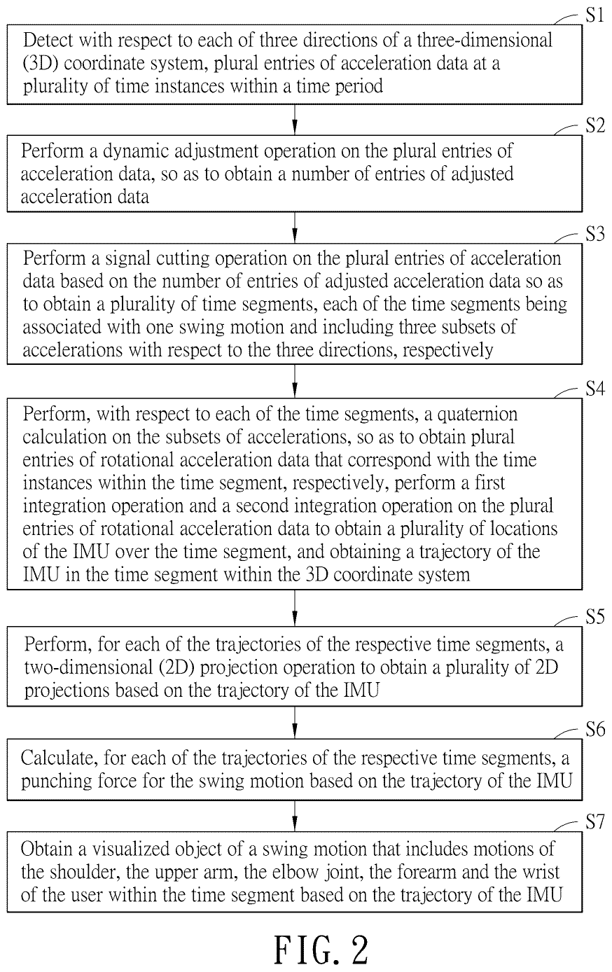

FIG. 2 is a flow chart illustrating steps of an exemplary method for implementing boxing motion analysis according to one embodiment of the disclosure. In this embodiment, the method is implemented using the system as shown in FIG. 1.

In step S1, the IMU 1 placed near the wrist of the user is activated to detect plural entries of acceleration data. Specifically, in embodiments, the IMU 1 detects, with respect to each of three directions of a three-dimensional (3D) coordinate system (e.g., an X direction, a Y direction and a Z direction in a 3D Cartesian system), plural entries of acceleration data at a plurality of time instances within a time period. It is noted that during the time period, the user may execute a plurality of swing motions. Each of the swing motions may be associated with a specific kind of punch, such as a jab, a cross, a hook, an uppercut, etc.

In this embodiment, the plural entries of acceleration data may be represented as αₓ(t), α_{y}(t) and α_{z}(t), where α represents the acceleration, x, y and z represent the three directions, respectively, and (t) represents a specific time instance. The plural entries of acceleration data are then transmitted to the computer device 3 via the communication unit 2.

In step S2, the processor 32 performs a dynamic adjustment operation on the plural entries of acceleration data, so as to obtain a number of entries of adjusted acceleration data.

Specifically, in this embodiment, the dynamic adjustment operation includes implementing matrix operations on the plural entries of acceleration data using the following equation:

$${A}_{t}^{O} = [ \begin{matrix}{k}_{x} & 0 & 0 \\ 0 & {k}_{y} & 0 \\ 0 & 0 & {k}_{z}\end{matrix} ] [ \begin{matrix}1 & - {θ}_{yz} & {θ}_{zy} \\ 0 & 1 & - {θ}_{zx} \\ 0 & 0 & 1\end{matrix} ] ( {A}_{t}^{s} - [ \begin{matrix}{b}_{x} & 0 & 0 \\ 0 & {b}_{y} & 0 \\ 0 & 0 & {b}_{z}\end{matrix} ] )$$

where

$${A}_{t}^{O}$$

represents a set of three entries of adjusted acceleration data with respect to the three directions at the time instance t, kₓ, k_{y} and k_{z} represent three scale factors with respect to the three directions, respectively, θ_{yz}, θ_{zy} and θ_{zx} represent three inclination factors each with respect to two of the three directions, bₓ, by and by represent three deviation factors with respect to the three directions, respectively, and

$${A}_{t}^{s}$$

represents a set of three entries of acceleration data with respect to the three directions at the time instance t.

In use, the scale factors, the inclination factors and the deviation factors may be calculated using the following equations:

$$G ( X ) = {\sum }_{t = 1}^{L} {( {‖ h ( {A}_{t}^{s} , X ) ‖}^{2} - {‖ {V}_{t} ‖}^{2} )}^{2} ,$$

$$X = [ {k}_{x} {k}_{y} {k}_{z} {θ}_{y z} {θ}_{z y} {θ}_{z x} {b}_{x} {b}_{y} {b}_{z} ]$$

where Vₜ represents an ideal dataset that is obtained using a motion capture system (e.g., one developed by Vicon) and that includes a set of 3D accelerations and a set of 3D angular velocities, L represents a count of data points detected by the IMU 1, and h represents the function

$$h ( {A}_{t}^{s} , X ) = {A}_{t}^{O} = [ \begin{matrix}{k}_{x} & 0 & 0 \\ 0 & {k}_{y} & 0 \\ 0 & 0 & {k}_{z}\end{matrix} ] [ \begin{matrix}1 & - {θ}_{yz} & {θ}_{zy} \\ 0 & 1 & - {θ}_{zx} \\ 0 & 0 & 1\end{matrix} ] ( {A}_{t}^{s} - [ \begin{matrix}{b}_{x} & 0 & 0 \\ 0 & {b}_{y} & 0 \\ 0 & 0 & {b}_{z}\end{matrix} ] )$$

The scale factors, the inclination factors and the deviation factors, which are included in the matrix X, are calculated by solving the equations such that the value of the function G(X) is a minimum. The calculation may be done using, for example, the Newton-Raphson method. It is noted that in some embodiments, other dynamic adjustments may be used to obtain the number of entries of adjusted acceleration data.

After the dynamic adjustment operation is completed, in step S3, the processor 32 implements a signal cutting operation on the plural entries of acceleration data recorded within the time period based on the number of entries of adjusted acceleration data, three average acceleration values corresponding respectively to the three directions when the IMU 1 is in a stationary state, and three threshold values.

Specifically, based on the number of entries of adjusted acceleration data, the processor 32 is configured to determine the time instances in which the IMU 1 is in a stationary state when the number of entries of adjusted acceleration data satisfy the following conditions:

$$\{ \begin{matrix}\vert {α}_{x}^{′} ( t ) - {I}_{x} \vert ≦ {Th}_{x} \\ \vert {α}_{y}^{′} ( t ) - {I}_{y} \vert ≦ {Th}_{y} \\ \vert {α}_{z}^{′} ( t ) - {I}_{z} \vert ≦ {Th}_{z}\end{matrix} ,$$

$$[ {I}_{x} {I}_{y} {I}_{z} ] = [ \frac{1}{N} {\sum }_{j = 1}^{N} {α}_{x}^{s} ( j ) , \frac{1}{N} {\sum }_{j = 1}^{N} {α}_{y}^{s} ( j ) , \frac{1}{N} {\sum }_{j = 1}^{N} {α}_{z}^{s} ( j ) ] ,$$

where α′ₓ(t), α′_{y}(t) and a′_{z}(t) represent three entries of adjusted acceleration data with respect to the three directions at the time instance t, respectively, Thₓ, Th_{y} and Th_{z} represent the three threshold values, respectively, and may be constants (e.g., 0.6 or other numbers), Iₓ, I_{y} and I_{z} each represent a respective one of the three average acceleration values of a number N of sample points at which the IMU 1 is in the stationary state, respectively, and the number N may be no smaller than 60. In each of the N sample points, the three entries of acceleration data with respect to the three directions are represented using

$${α}_{x}^{s} , {α}_{y}^{s} and {α}_{z}^{s} ,$$

respectively.

With the stationary state defined, the processor 32 is configured to determine, during the time period, at which time instance(s) the IMU 1 is not in the stationary state, using the following conditions:

$$\{ \begin{matrix}\vert {α}_{x} ( t ) - {I}_{x} \vert > {Th}_{x} \\ \vert {α}_{y} ( t ) - {I}_{y} \vert > {Th}_{y} \\ \vert {α}_{z} ( t ) - {I}_{z} \vert > {Th}_{z}\end{matrix} .$$

Afterwards, the processor 32 is configured to “trim” the plural entries of acceleration data recorded within the time period to obtain a plurality of time segments defined by the time instances at which the IMU 1 is not in the stationary state, each time segment being associated with one swing motion and including three subsets of accelerations with respect to the three directions, respectively.

Then, in step S4, the processor 32 performs, with respect to each of the time segments, a quaternion calculation on the subsets of accelerations, so as to obtain plural entries of rotational acceleration data that correspond with the time instances within the time segment, respectively. Then, the processor 32 performs a first integration operation on the plural entries of rotational acceleration data to obtain a plurality of velocities of the IMU 1 over the time segment, and performs a second integration operation on the plural entries of rotational acceleration data to obtain a plurality of locations of the IMU 1 over the time segment. As such, a trajectory of the IMU 1 in the time segment, i.e., a trajectory of the corresponding swing motion, within the 3D coordinate system may be obtained.

In embodiments, the quaternion calculation on the subsets of accelerations may be done using the following equations:

$${a}_{t}^{rot} = {Q}_{t} ⊗ {a}_{t}^{c a l} ⊗ \overline{{Q}_{t}} ,$$

$${Q}_{t} = {Q}_{t - 1} + \dot{Q} ,$$

$$\dot{Q} = \frac{1}{2} \vert \begin{matrix}0 & - {ω}_{x} & - {ω}_{y} & - {ω}_{z} \\ {ω}_{x} & 0 & - {ω}_{z} & - {ω}_{y} \\ {ω}_{y} & - {ω}_{z} & 0 & {ω}_{x} \\ {ω}_{z} & {ω}_{y} & - {ω}_{x} & 0\end{matrix} \vert$$

where

$${a}_{t}^{rot}$$

represents a set of three entries of rotational acceleration data with respect to the three directions at the time instance t, respectively,

$${a}_{t}^{cal}$$

represents a set of three entries of adjusted acceleration data with respect to the three directions at the time instance t, respectively, Qₜ represents a quaternion associated with the time instance t, ⊗ represents a tensor product operation, Qₜ₋₁ represents a quaternion associated with the time instance t−1, (i.e., the immediate preceding time instance), and ωₓ, ω_{y} and ω_{z} represent the angular velocities of the IMU 1 with respect to the three directions at the time instance t, respectively.

After the trajectory of the IMU 1 in each of the time segments is obtained, in step S5, the processor 32 performs, for each of the trajectories of the respective time segments, a two-dimensional (2D) projection operation to obtain a plurality of 2D projections based on the trajectory of the IMU 1. Specifically, in this embodiment, the 2D projections may be in the form of images with 50*50 pixels, and may correspond with different planes within the 3D coordinate systems. For example, in this embodiments, four 2D projections may be obtained, with three of the four 2D projections corresponding with the x-y plane, the y-z plane, the x-z plane and an arbitrary plane within the 3D coordinate system.

Then, the processor 32 combines the 2D projections to create a composite image. In this embodiment, the composite image may be in the size of 100*100 pixels. The composite image may be then inputted into an image recognition model (e.g., MobileNet), so as to determine the specific kind of punch associated with the swing motion.

Then, in step S6, the processor 32 calculates, for each of the trajectories of the respective time segments, a punching force for the swing motion based on the trajectory of the IMU 1. Specifically, the punching force may be calculated using the following equation:

$${F}_{t} = {M}_{t} \times {‖ {a}_{t}^{cal} ‖}_{2} , \{ \text{} \begin{matrix}{D}_{t} \leq {L}_{1} , {M}_{t} = \frac{{D}_{t}}{{L}_{1}} \times {m}_{1} + {m}_{3} + {m}_{4} \\ {D}_{t} > {L}_{1} , {M}_{t} = \frac{{D}_{t} - {L}_{1}}{{L}_{2}} \times {m}_{2} + {m}_{1} + {m}_{3} + {m}_{4}\end{matrix} \text{}$$

where Fₜ represents the punching force at the time instance

$$t , ‖ {a}_{t}^{cal} ‖$$

represent three entries of adjusted acceleration data with respect to the three directions at the time instance t, respectively, Dₜ represents a punching distance between an initial location of the IMU 1 and the location of the IMU 1 at the time instance t, L₁ represents a length of the forearm of the user, L₂ represents a length of the upper arm of the user, m₁ represents a weight of the forearm of the user, m₂ represents a weight of the upper arm of the user, m₃ represents a weight of the hand of the user, and m₄ represents a weight of the wearable device 9.

It is noted that as the relation between the punching distance and the length of the forearm of the user changes, different equations are used for determining the punching force. For example, in the case that the punching distance is smaller than the length of the forearm of the user, when calculating the punching force, a percentage of the weight of the forearm is first calculated based on the punching distance and the length of the forearm, and the weight of the upper arm is not taken into consideration. On the other hand, in the case that the punching distance is larger than the length of the forearm of the user, when calculating the punching force, the weight of the entire forearm and a percentage of the weight of the upper arm, which is calculated based on the punching distance, the length of the forearm and the length of the upper arm are taken into consideration.

It is noted that various pieces of information related to the user such as a height, a weight, the length of the forearm of the user, the length of the upper arm of the user, the weight of the forearm of the user, the weight of the upper arm of the user, the weight of the hand of the user, and the weight of the wearable device 9 may be pre-stored in the data storage unit 34. In other embodiments, the data storage unit 34 may store only the height and the weight of the user and the weight of the wearable device 9, and other information may be estimated using, for example, a lookup table or a set of equations.

Then, in step S7, the processor 32, for each of the trajectories of the respective time segments and with respect to each of the plurality of time instances within the time segment, defines the location of the IMU 1 as a first center point (which serves as a location of the wrist of the user), and an initial location of the IMU 1 as a second center point (which serves as a location of a shoulder of the user). Then, the processor 32 obtains a first circle using the first center point as a center, and the length of the forearm of the user as a radius, and obtains a second circle using the second center point as a center, and the length of the upper arm of the user as a radius. In this manner, two intersecting circles are created with two intersections, and one of the two intersections with a lower height (i.e., with a smaller Z-coordinate value) may be designated as a location of an elbow joint of the user. As such, the locations of the shoulder, the upper arm, the elbow joint, the forearm and the wrist of the user may be obtained for a time instance. Then, by combining the above information in different time instances, a visualized object of a swing motion that includes motions of the shoulder, the upper arm, the elbow joint, the forearm and the wrist of the user within the time segment may be obtained. In embodiments, the visualized object may be images, an animation, computer graphics, etc., and creating the visualized object may be implemented by using algorithms and/or software available on the market. For example, the computer device 3 may execute a digital human modelling (DHM) software to create an arm model of the user, and based on the motions of the shoulder, the upper arm, the elbow joint, the forearm and the wrist of the user within the time segment, an arm model with movement may be created and outputted by a computer. As such, the user may be able to view the movement of his/her own arm during the swing motion. The arm model with movement may be further used to determine whether the swing motion is appropriate.

It is noted that in different embodiments, the operations of steps S5, S6 and S7 may be done in any given order, and is not limited to the depiction of FIG. 2. In some embodiments, some or all of the operations of steps S5, S6 and S7 may be omitted.

While in this embodiment the method may be done in a real time manner, meaning the plural entries of acceleration data are immediately transmitted to the computer device 3 for processing, in some embodiments, the wearable device 9 may not have a wireless communication function, and the plural entries of acceleration data may be stored in a data storage of the wearable device 9, and obtained and processed by the computer device 3 afterwards.

While in this embodiment only one IMU 1 is involved and is worn on one wrist of the user, in other embodiments, two IMUs 1 may be worn on both the two wrists of the user respectively to obtain even more information.

To sum up, the embodiments of the disclosure provide a method and a system for implementing boxing motion analysis. In the method, after receiving the plural entries of acceleration data from the IMU 1, the computer device 3 is configured to perform a dynamic adjustment operation on the plural entries of acceleration data, so as to obtain a number of entries of adjusted acceleration data, and to implement a signal cutting operation based on the number of entries of adjusted acceleration data, so as to obtain a plurality of time segments, each being associated with one swing motion. Then, the computer device performs, with respect to each of the time segments, a quaternion calculation on the subsets of accelerations, so as to obtain plural entries of rotational acceleration data that correspond with the time instances within the time segment, respectively. Then, the computer device 3 performs a first integration operation and a second integration operation to obtain a plurality of locations of the IMU 1 over the time segment. As such, a trajectory of the IMU 1 in the time segment within the 3D coordinate system may be obtained. Using the trajectories of the IMU 1 in the respective time segments, further analysis may be performed, such as determining a type of punch, calculating a punching force or generating a visualized object of a punching motion. As such, the above effects may be achieved using only one IMU.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for implementing boxing motion analysis, the method being implemented using an inertial measurement unit (IMU) placed on a wrist of a user, and a computer device, the method comprising:
a) obtaining, by the computer device from the IMU, plural entries of acceleration data at a plurality of time instances within a time period, each of the plural entries of acceleration data being with respect to one of three directions of a three-dimensional (3D) coordinate system;
b) performing a dynamic adjustment operation on the plural entries of acceleration data, so as to obtain a number of entries of adjusted acceleration data;
c) performing a signal cutting operation on the plural entries of acceleration data based on the number of entries of adjusted acceleration data, three average acceleration values corresponding respectively to the three directions when the IMU is in a stationary state, and three threshold values, so as to obtain a plurality of time segments, each of the time segments being associated with one swing motion and including three subsets of accelerations with respect to the three directions, respectively; and
d) performing, with respect to each of the time segments, a quaternion calculation on the subsets of accelerations, so as to obtain plural entries of rotational acceleration data that correspond with the time instances within the time segment, respectively, performing a first integration operation on the plural entries of rotational acceleration data to obtain a plurality of velocities of the IMU over the time segment, performing a second integration operation on the plural entries of rotational acceleration data to obtain a plurality of locations of the IMU over the time segment, and obtaining a trajectory of the IMU in the time segment within the 3D coordinate system.

2. The method as claimed in claim 1, wherein step c) includes
determining the time instances in which the IMU is in the stationary state when the number of entries of adjusted acceleration data satisfy the following conditions:
$$\text{} \{ \begin{matrix}\vert {α}_{x} ( t ) - {I}_{x} \vert ≦ {Th}_{x} \\ \vert {α}_{y} ( t ) - {I}_{y} \vert ≦ {Th}_{y} \\ \vert {α}_{z} ( t ) - {I}_{z} \vert ≦ {Th}_{z}\end{matrix} ,$$
$$[ \begin{matrix}{I}_{x} & {I}_{y} & {I}_{z}\end{matrix} \text{} ] = [ \frac{1}{N} {\sum }_{j = 1}^{N} {α}_{x}^{s} ( j ) , \frac{1}{N} {\sum }_{j = 1}^{N} {α}_{y}^{s} ( j ) , \frac{1}{N} {\sum }_{j = 1}^{N} {α}_{z}^{s} ( j ) ] ,$$
where αₓ(t), α_{y}(t) and α_{z}(t) represent three entries of acceleration data with respect to the three directions at the time instance t, respectively, Thₓ, Th_{y} and Th_{z} represent the three threshold values, respectively, Iₓ, I_{y} and I_{z} each represent a respective one of the three average acceleration values of a number N of sample points involving the stationary state, respectively; and
determining, during the time period, at which time instance(s) the IMU is not in the stationary state, using the following conditions,
$$\text{} \{ \begin{matrix}\vert {α}_{x} ( t ) - {I}_{x} \vert > {Th}_{x} \\ \vert {α}_{y} ( t ) - {I}_{y} \vert > {Th}_{y} \\ \vert {α}_{z} ( t ) - {I}_{z} \vert > {Th}_{z}\end{matrix} ,$$
so as to “trim” the plural entries of acceleration data recorded within the time period to obtain a plurality of time segments defined by the time instances at which the IMU is not in the stationary state, each time segment being associated with one swing motion and including three subsets of accelerations with respect to the three directions, respectively.

3. The method as claimed in claim 1, wherein step c) includes:
implementing the quaternion calculation on the subsets of accelerations using the following equations:
$${a}_{t}^{rot} = {Q}_{t} ⊗ {a}_{t}^{cal} ⊗ \overline{{Q}_{t} ,} {Q}_{t} = {Q}_{t - 1} + \dot{Q} , \dot{Q} = \frac{1}{2} \vert \text{} \begin{matrix}0 & - {ω}_{x} & - {ω}_{y} & - {ω}_{z} \\ {ω}_{x} & 0 & - {ω}_{z} & - {ω}_{y} \\ {ω}_{y} & - {ω}_{z} & 0 & {ω}_{x} \\ {ω}_{z} & {ω}_{y} & - {ω}_{x} & 0\end{matrix} \vert \text{}$$
where
$${a}_{t}^{rot}$$
represents a set of three entries of rotational acceleration data with respect to the three directions at the time instance t, respectively,
$${a}_{t}^{c a l}$$
represents a set of three entries of adjusted acceleration data with respect to the three directions at the time instance t, respectively, Qₜ represents a quaternion associated with the time instance t, ⊗ represents a tensor product operation, Qₜ₋₁ represents a quaternion associated with the time instance t−1, (i.e., the immediate preceding time instance), and ωₓ, ω_{y} and ω_{z} represent the angular velocities of the IMU with respect to the three directions at the time instance t, respectively.

4. The method as claimed in claim 1, further comprising:
e) performing, for each of the trajectories of the respective time segments, a two-dimensional (2D) projection operation to obtain a plurality of 2D projections based on the trajectory of the IMU, combining the 2D projections to create a composite image, and inputting the composite image into an image recognition model so as to determine a kind of punch associated with the swing motion.

5. The method as claimed in claim 1, further comprising, after step d):
f) calculating, for each of the trajectories of the respective time segments, a punching force for the swing motion based on the trajectory of the IMU using the following equation:
$${F}_{t} = {M}_{t} \times ‖ {a}_{t}^{cal} ‖ , \{ \begin{matrix}{D}_{t} \leq {L}_{1} , {M}_{t} = \frac{{Q}_{t}}{{L}_{1}} \times {m}_{1} + {m}_{3} + {m}_{4} \\ {D}_{t} > {L}_{1} , {M}_{t} = \frac{{Q}_{t} - {L}_{1}}{{L}_{2}} \times {m}_{2} + {m}_{1} + {m}_{3} + {m}_{4}\end{matrix} \text{}$$
where Fₜ represents the punching force at the time instance
$$t , ‖ {a}_{t}^{cal} ‖$$
represent three entries of adjusted acceleration data with respect to the three directions at the time instance t, Dₜ represents a punching distance between an initial location of the IMU and the location of the IMU at the time instance t, L₁ represents a length of the forearm of the user, L₂ represents a length of the upper arm of the user, m₁ represents a weight of the forearm of the user, m₂ represents a weight of the upper arm of the user, m₃ represents a weight of the hand of the user, and m₄ represents a weight of the wearable device.

6. The method as claimed in claim 5, the computer storing a height and a weight of the user, wherein step f) includes estimating the length of the forearm of the user, the weight of the forearm of the user, the weight of the upper arm of the user, and the weight of the hand of the user, using a lookup table.

7. The method as claimed in claim 1, further comprising, after step d):
for each of the trajectories of the respective time segments and with respect to each of the plurality of time instances within the time segment, defining the location of the IMU as a first center point which serves as a location of the wrist, and an initial location of the IMU as a second center point which serves as a location of a shoulder of the user;
obtaining a first circle using the first center point as a center, and a length of a forearm of the user as a radius, and obtaining a second circle using the second center point as a center, and a length of an upper arm of the user as a radius, thus creating two intersecting circles with two intersections, and designating one of the two intersections with a lower height as a location of an elbow joint of the user;
using the locations of the shoulder, the elbow joint and the wrist of the user to obtain a visualized object of a swing motion.

8. The method as claimed in claim 1, wherein the dynamic adjustment operation includes implementing matrix operations on the plural entries of acceleration data using the following equation:
$${A}_{t}^{O} = [ \text{} \begin{matrix}{k}_{x} & 0 & 0 \\ 0 & {k}_{y} & 0 \\ 0 & 0 & {k}_{z}\end{matrix} \text{} ] [ \text{} \begin{matrix}1 & - {θ}_{yz} & {θ}_{zy} \\ 0 & 1 & - {θ}_{zx} \\ 0 & 0 & 1\end{matrix} \text{} ] ( {A}_{t}^{s} - [ \begin{matrix}{b}_{x} & 0 & 0 \\ 0 & {b}_{y} & 0 \\ 0 & 0 & {b}_{z}\end{matrix} \text{} ] )$$
where
$${A}_{t}^{O}$$
represents a set of three entries of adjusted acceleration data with respect to the three directions at the time instance t, kₓ, k_{y} and k_{z} represent three scale factors with respect to the three directions, respectively, θ_{yz}, θ_{zy} and θ_{zx} represent three inclination factors each with respect to two of the three directions, bₓ, b_{y} and b_{y} represent three deviation factors with respect to the three directions, respectively, and
$${A}_{t}^{s}$$
represents a set of three entries of acceleration data with respect to the three directions at the time instance t.

9. A system implementing boxing motion analysis, the system comprising an inertial measurement unit (IMU) placed on a wrist of a user and a computer device connected to the IMU, wherein the computer device is programmed to:
obtain from the IMU plural entries of acceleration data at a plurality of time instances within a time period, each of the plural entries of acceleration data being with respect to one of three directions of a three-dimensional (3D) coordinate system;
perform a dynamic adjustment operation on the plural entries of acceleration data, so as to obtain a number of entries of adjusted acceleration data, wherein the dynamic adjustment operation includes implementing matrix operations on the plural entries of acceleration data using the following equation
$${A}_{t}^{O} = [ \text{} \begin{matrix}{k}_{x} & 0 & 0 \\ 0 & {k}_{y} & 0 \\ 0 & 0 & {k}_{z}\end{matrix} \text{} ] [ \text{} \begin{matrix}1 & - {θ}_{yz} & {θ}_{zy} \\ 0 & 1 & - {θ}_{zx} \\ 0 & 0 & 1\end{matrix} \text{} ] ( {A}_{t}^{s} - [ \begin{matrix}{b}_{x} & 0 & 0 \\ 0 & {b}_{y} & 0 \\ 0 & 0 & {b}_{z}\end{matrix} \text{} ] )$$
where
$${A}_{t}^{O}$$
represents a set of three entries of adjusted acceleration data with respect to the three directions at the time instance t, kₓ, k_{y} and k_{z} represent three scale factors with respect to the three directions, respectively, θ_{yz}, θ_{zy} and θ_{zx} represent three inclination factors each with respect to two of the three directions, bₓ, b_{y} and b_{y} represent three deviation factors with respect to the three directions, respectively, and
$${A}_{t}^{s}$$
represents a set of three entries of acceleration data with respect to the three directions at the time instance t;
perform a signal cutting operation on the plural entries of acceleration data based on the number of entries of adjusted acceleration data, three average acceleration values corresponding respectively to the three directions when the IMU is in a stationary state, and three threshold values, so as to obtain a plurality of time segments, each of the time segments being associated with one swing motion and including three subsets of accelerations with respect to the three directions, respectively; and
perform, with respect to each of the time segments, a quaternion calculation on the subsets of accelerations, so as to obtain plural entries of rotational acceleration data that correspond with the time instances within the time segment, respectively, performing a first integration operation on the plural entries of rotational acceleration data to obtain a plurality of velocities of the IMU over the time segment, performing a second integration operation on the plural entries of rotational acceleration data to obtain a plurality of locations of the IMU over the time segment, and obtaining a trajectory of the IMU in the time segment within the 3D coordinate system.
